(19) United States
(12) Patent Application Publication
(10) Pub. No.: US 2025/0134580 A1
(43) Pub. Date: May 1, 2025

(54) BIPOLAR ELECTRODE FOR A RESECTOSCOPE AND RESECTOSCOPE WITH SUCH A BIPOLAR ELECTRODE

(71) Applicant: Henke-Sass, Wolf GmbH, Tuttlingen (DE)

(72) Inventors: Armin PFAFF, Dunningen (DE); Stefan RAPP, Donaueschingen (DE)

(21) Appl. No.: 18/930,696

(22) Filed: Oct. 29, 2024

(30) Foreign Application Priority Data
Oct. 30, 2023 (DE) 102023129912.3

(51) Int. Cl. A61B 18/14; A61B 18/00; A61B 18/12

(52) U.S. Cl. CPC A61B 18/14; A61B 2018/00077; A61B 2018/00083; A61B 2018/00148; A61B 2018/00178; A61B 2018/126; A61B 2018/144

(57) ABSTRACT

A bipolar electrode for a resectoscope includes a proximal contact portion, a middle portion adjoining the proximal contact portion and a distal end portion adjoining the middle portion. The bipolar electrode includes an active electrode and a neutral electrode. The neutral electrode has a hollow cylindrical shape in the middle portion and coaxially surrounds the active electrode. The proximal contact portion includes a first contact of the active electrode and a second contact of the neutral electrode. The neutral electrode is surrounded by a first insulation from the proximal contact portion to the distal end portion. Both a first end region of the active electrode and a second end region of the neutral electrode are exposed at the distal end portion, so that an electrical voltage applied via the contacts is applied directly between the two end regions.

### PRIORITY

This application claims the benefit of German Patent Application No. 10 2023 129 912.3, filed on Oct. 30, 2023, which is hereby incorporated herein by reference in its entirety.

### FIELD

The present invention relates to a bipolar electrode for a resectoscope and a resectoscope with such a bipolar electrode.

### BACKGROUND

Known resectoscopes with a bipolar electrode are designed in such that the neutral electrode of the bipolar electrode is connected via the resectoscope itself or via a separate contact on the patient. This can make it difficult to reliably ignite the desired plasma at the distal end of the bipolar electrode for the treatment.

### SUMMARY

It is an object herein to provide an improved bipolar electrode for a resectoscope and a resectoscope with such a bipolar electrode.

The bipolar electrode for a resectoscope may comprise a proximal contact portion, a middle portion adjoining the proximal contact portion and a distal end portion adjoining the middle portion, wherein the bipolar electrode comprises an active electrode and a neutral electrode. The neutral electrode can have a hollow cylindrical shape in the middle portion and coaxially surround the active electrode. The proximal contact portion may comprise a first contact of the active electrode and a second contact of the neutral electrode, wherein the neutral electrode is surrounded by a first insulation from the proximal contact portion to the distal end portion. At the distal end portion, a first end region of the active electrode and a second end region of the neutral electrode are exposed, so that an electrical voltage applied via the contacts is applied directly between the two end regions.

This ensures that the desired plasma is reliably ignited during treatment.

The active electrode can comprise a copper conductor in the middle section. With such a copper conductor, the necessary high currents can be safely conducted without causing temperature problems during treatment.

The active electrode can comprise a hollow cylindrical conductor in the middle section. A conductive wire can be arranged in the hollow cylindrical conductor. In particular, the hollow cylindrical conductor can be made of copper.

Furthermore, the active electrode can comprise a solid conductor in the middle portion. This means in particular that the conductor is not hollow.

Furthermore, the active electrode can comprise an element for mechanical reinforcement in the area of the proximal contact portion. In particular, the element for mechanical reinforcement can have a cylindrical shape and can be inserted in the hollow cylindrical conductor of the active electrode. The element for mechanical reinforcement can be made of tungsten, stainless steel, spring steel, etc. Furthermore, a second insulation can be provided between the active electrode and the neutral electrode.

Furthermore, the distal end portion may be fork-shaped with a first fork arm and a second fork arm, wherein the exposed first end region of the active electrode is located between the two fork arms.

Each fork arm may comprise a third insulation, which is arranged between the active electrode and the neutral electrode and overlaps with the second insulation.

The exposed first end region of the active electrode can be formed as a tungsten wire. However, it is also possible for the exposed first end region of the active electrode to be formed from stainless steel.

The neutral electrode can comprise a hollow cylindrical conductor in the middle portion. In particular, the conductor can be made of stainless steel.

The first contact of the active electrode and/or the second contact of the neutral electrode may comprise a metallic sleeve. The metallic sleeve can be made of a metallic conductor, such as stainless steel.

The first contact of the active electrode and/or the second contact of the neutral electrode may comprise a metallic coating. The metallic coating can be gold, silver, aluminum or zinc, for example. The metallic coating can provide a better electrical contact and/or improved corrosion resistance.

The second contact of the neutral electrode can be designed such that the second contact can be brought into engagement with a snap-in connection.

The proximal contact portion of the bipolar electrode may comprise an outer diameter in the range of 0.8-1.2 mm and in particular an outer diameter of 1.0 mm.

The outer diameter of the middle portion of the bipolar electrode can be in the range of 1.6-2.0 mm and in particular can be 1.8 mm.

The length of the bipolar electrode can be in the range of 200-400 mm, in particular in the range of 250-350 mm and furthermore in particular in the range of 280-300 mm.

Each of the first, second and third insulations can be designed as a shrink sleeve. In particular, a fluoropolymer shrink tube, e.g. a PTFE shrink tube (polytetrafluoroethylene shrink tube) or a PVDF shrink tube (polyvinylidene fluoride shrink tube) can be used.

The bipolar electrode can thus be described as a coaxial bipolar electrode due to its structure, since the neutral electrode runs coaxially to the active electrode in the middle portion. Furthermore, both electrodes (neutral electrode and active electrode) are guided from the proximal contact portion to the distal end, so that the current can flow between the two exposed end regions during operation. Therefore, advantageously, when the present bipolar electrode is used as intended, no current needs to be conducted to or from the patient. The full potential difference (or voltage), which is applied to the proximal contact portion of the bipolar electrode, is present at the two end regions and thus at the distal end of the bipolar electrode. This ensures good ignition behavior for the desired plasma, e.g. in a NaCl solution.

The bipolar electrode is designed in particular for a voltage of 90 volts-4000 volts (especially 90 volts-1500 volts or 200 volts-1500 volts) at a frequency of 300 kHz to 4 MHz.

The first end region of the active electrode can be designed as a loop, cone, knife, roller, etc. This makes it possible to cut and/or coagulate the corresponding tissue in the area of the distal end of the bipolar electrode when a corresponding electrical voltage is applied via the contacts.

Furthermore, a resectoscope with a bipolar electrode is provided.

The resectoscope may comprise a guide block. A latching element can be provided in the guide block, which in the inserted state of the bipolar electrode effects a latching connection with the second contact of the neutral electrode. Alternatively, the latching element can effect a latching connection with the first contact of the active electrode when the bipolar electrode is inserted. The first or second contact is preferably designed in such a way that the desired latching connection is present (preferably by means of positive locking). The latching or snap-in connection can be designed in such a way that the bipolar electrode is mechanically fixed in the guide block and any displacement of the guide block also moves the bipolar electrode.

The guide block can be designed in such a way that the bipolar electrode is detachably fixed.

The guide block can be displaceable in the longitudinal direction of the resectoscope.

Furthermore, a first contact section for the first contact of the active electrode and a second contact section for the second contact of the neutral electrode can be provided in the guide block, wherein a seal is arranged between the two contact sections and wherein the seal, when the bipolar electrode is inserted, prevents liquid from reaching the second contact (or vice versa).

The first and/or second contact section can be formed from a metal, such as stainless steel. Furthermore, the first and/or second contact section may comprise a metallic coating. The metallic coating may be gold, silver, aluminum or zinc, for example. The metallic coating can provide a better electrical contact and/or improved corrosion resistance.

The resectoscope may comprise other elements known to the skilled person.

It is to be understood that the above-mentioned features and those to be explained below can be used not only in the combinations indicated, but also in other combinations or on their own, without going beyond the scope of the present invention.

In the following, the invention is explained in more detail with reference to the attached drawings, which also disclose features essential to the invention. These embodiments are for illustrative purposes only and are not to be construed as limiting. For example, a description of an embodiment with a plurality of elements or components is not to be interpreted as meaning that all of these elements or components are necessary for implementation. Rather, other embodiments may include alternative elements and components, fewer elements or components, or additional elements or components. Elements or components of different embodiments may be combined with each other, unless otherwise indicated. Modifications and variations described for one of the embodiments may also be applicable to other embodiments. To avoid repetition, identical or corresponding elements in different figures are designated with the same reference signs and are not explained more than once.

### BRIEF DESCRIPTION OF THE DRAWINGS

FIG. 1 is a schematic perspective view of an embodiment of the resectoscope in accordance with the invention

FIG. 2 is a side view of the bipolar electrode in accordance with the invention.

FIG. 3 is a view of the distal end portion of the bipolar electrode.

FIG. 4 is an enlarged view of the detail B of FIG. 2 in a section A-A according to FIG. 3.

FIG. 5 is a sectional view along the sectional line C-C according to FIG. 3 through the guide block with inserted bipolar electrode.

FIG. 6 is a sectional view along the sectional line A-A according to FIG. 3 through the guide block with inserted bipolar electrode.

FIG. 7 is a top view of the distal end portion of the bipolar electrode.

FIG. 8 is a sectional view along the sectional line C-C according to FIG. 3 of the area E in FIG. 7.

FIG. 9 is an enlarged view of the detail F of FIG. 8.

FIG. 10 is an enlarged view of the detail G of FIG. 8.

FIG. 11 is a sectional view along the sectional line D-D according to FIG. 3 of the fork tube.

### DETAILED DESCRIPTION

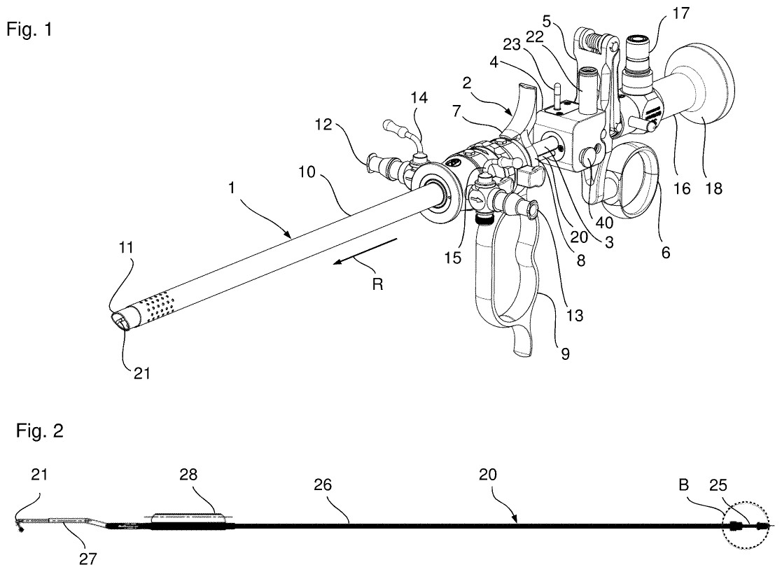

In the embodiment shown in FIG. 1, the resectoscope 1 comprises a working element 2 with an optical tube 3 on which a guide block 4 is mounted so as to be displaceable in the longitudinal direction R. The guide block 4 is held in the first position shown in FIG. 1 by means of a spring element 5. A thumb ring 6 is formed on the spring element 5. Furthermore, the working element 2 comprises a front section 7 spaced from the guide block 4 in its first position in the longitudinal direction R, which comprises a stop 8 and a finger grip 9. The front section 7 is firmly connected to the optical tube 2 in such a way that no movement in the longitudinal direction R is possible.

Furthermore, the resectoscope 1 comprises an irrigation shaft 10 which is detachably fastened to an end of the front section 7 pointing away from the guide block 4 and in which the optical tube 3 runs up to a distal end 11 of the irrigation shaft 10 and is thus surrounded by the irrigation shaft 10. The irrigation shaft 10 comprises an inflow connection 12 and an outflow connection 13, each of which can be shut off by a valve 14, 15.

At an end of the working element 2 pointing away from the distal end 11 of the irrigation shaft 10, an observation optic 16 is detachably connected, the endoscope observation optic of which, not shown in FIG. 1, extends through the optical tube 3 to the distal end 11 in a manner known from resectoscopes. FIG. 1 shows an optical fiber connection 17 and an insight 18.

Furthermore, the resectoscope 1 comprises a bipolar electrode 20, which is detachably fixed in the guide block 4, as will be described in detail below. The bipolar electrode 20 extends through the irrigation shaft 10 to the distal end 11 and does not protrude beyond the distal end 11 in the position of the guide block 4 shown in FIG. 1. A distal end 21 of the electrode 20 is thus still within the irrigation shaft 10. However, when the guide block 4 is moved by an operator in the direction towards the stop 8 of the front section 7, the bipolar electrode 20 fixed in the guide block 4 is thereby also moved in this direction and thus in the longitudinal direction R, whereby the distal end 21 of the bipolar electrode 20 is moved beyond the distal end 11 of the irrigation shaft 10.

However, it is also possible that in the position of the guide block 4 shown in FIG. 1, the distal end 21 of the electrode 20 protrudes beyond the distal end 11 of the irrigation shaft 10. If the guide block 4 is then moved by an operator in the direction of the stop 8 of the front section 7, the bipolar electrode 20 fixed in the guide block 4 is thereby also moved in this direction and thus in the longitudinal direction R, whereby the distal end 21 of the bipolar electrode 20 is moved further beyond the distal end 11 of the irrigation shaft 10.

The guide block 4 comprises a first electrical connection 22 for an active electrode 34 of the bipolar electrode 20 and a second electrical connection 23 for a neutral electrode 38 of the bipolar electrode 20.

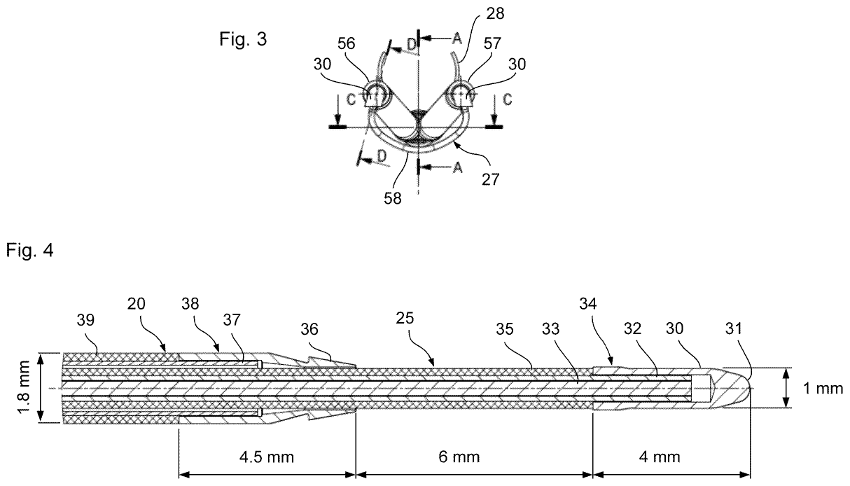

As can best be seen in FIG. 2, the bipolar electrode 20 comprises a proximal contact portion 25, a middle portion 26 adjoining the proximal contact portion 25 and a distal end portion 27 adjoining the middle portion 26. A guide 28 is formed on the middle portion 26, which rests against the optical tube 3 and supports the movement of the bipolar electrode 20 in the longitudinal direction R. In FIG. 3, a view of the distal end portion 27 of the bipolar electrode 20 is shown to explain the subsequent sectional views. Thus, the proximal contact portion 25 (detail B in FIG. 2) is shown enlarged in section A-A in FIG. 4.

An end sleeve 30 with a rounded proximal end 31 is formed at the proximal end of the contact portion 25, wherein the end sleeve 30 is made here of stainless steel and is pressed with an inner tube 32, which protrudes into the end sleeve 30 and which is preferably hollow-cylindrical in shape. The end sleeve 30 forms a first contact 30 of the active electrode 34. For its part, the inner tube 32 is provided with a cylindrical reinforcement 33, which rests in the inner tube 32 against the inner wall of the inner tube 32. This reinforcement 33 extends at least over the entire proximal contact portion 25. For example, tungsten, spring steel, wire, stainless steel, etc. can be used as the material for the reinforcement. The end sleeve 30 and the inner tube 32, which is preferably made of copper, are part of the active electrode 34 of the bipolar electrode 20. An insulation 35 is formed on the inner tube 32 in the longitudinal direction R adjacent to the end sleeve 30. The insulation 35 has preferably a hollow cylindrical shape and can, for example, be formed as a shrink tube (e.g. PTFE).

A latching sleeve 36 is arranged on the insulation 35 at a distance in the longitudinal direction R from the end sleeve 30, which is connected to a main tube 37 also arranged on the insulation 35. The latching sleeve 36 forms a second contact 36 of the neutral electrode 38. The latching sleeve 36 and the main tube 37 can each be made of stainless steel, for example. The latching sleeve 36 can be welded to the main tube 37. Laser welding can be used for this purpose, for example. The latching sleeve 36 and the main tube 37 are parts of the neutral electrode 38 of the bipolar electrode 20. An insulation 39, which is also referred to below as the first insulation 39, is formed on the main tube 37 in the longitudinal direction R adjacent to the latching sleeve 36. Furthermore, the insulation 35 between inner tube 32 and latching sleeve 36 or main tube 37 is also referred to below as second insulation 35. The first insulation 39 can be formed in the same way as the second insulation 35 as a heat-shrinkable tube and in particular as a PTFE tube. However, it is also possible for the first insulation 39 to be formed as a PVDF shrink tube.

The first and second insulations 39, 35 run from the contact portion 25 via the middle portion 26 to the distal end portion 27, with the second insulation 35 serving as insulation between the active electrode 34 and the neutral electrode 38 and the first insulation 39 serving to insulate the neutral electrode 38 from the environment.

As can be clearly seen in FIG. 4, the reinforcement 33 serves to mechanically stabilize the contact portion 25, particularly in the area between the end sleeve 30 and the latching sleeve 36, since otherwise only the hollow inner tube 32 would have to provide stabilization in this area, which would be difficult due to the small outer diameter of approx. 1 mm in the area of the end sleeve 30.

In the embodiment described here, the end sleeve 30 extends over a distance of 4 mm in the longitudinal direction R. Furthermore, the distance between the end sleeve 30 and the detent sleeve 36 is 6 mm and the latching sleeve 36 extends over a distance of 4.5 mm in the longitudinal direction R, so that the entire contact portion 25 comprises a length of 14.5 mm. The reinforcement 33 is approximately twice as long as the contact portion 25 and extends here in the longitudinal direction R over a distance of 30 mm.

As also shown in FIG. 4, the outer diameter of the first insulation 39 is 1.8 mm.

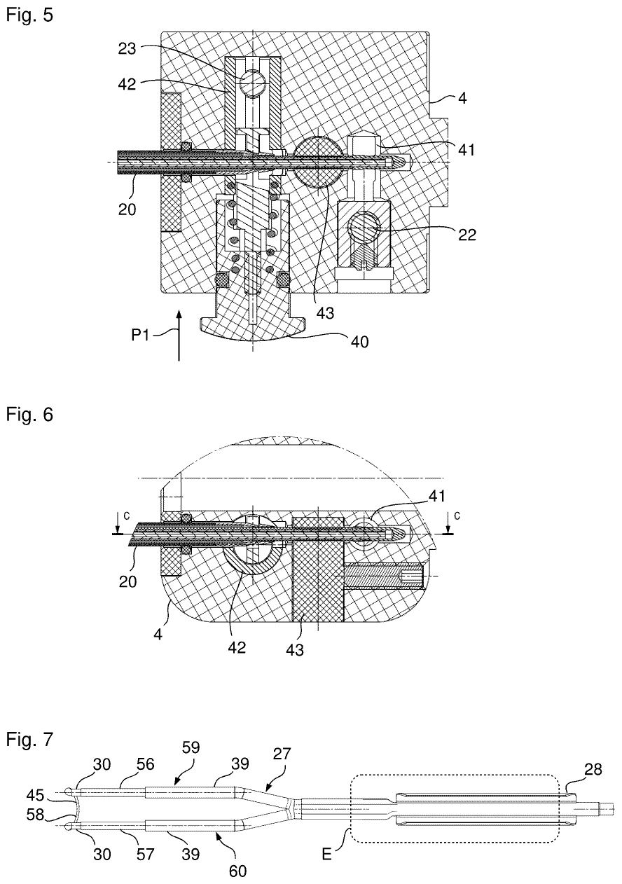

FIG. 5 shows the section C-C (FIG. 3) through the guide block 4 with inserted electrode 20 and FIG. 6 shows the section A-A (FIG. 3) through the guide block 4 with inserted electrode 20.

In the guide block 4 there is a spring-biased latching element 40, which is in engagement with the latching sleeve 36 and thus locks the electrode 20, whereby in this state the end sleeve 30 is in contact with a first contact section 41, which ensures the desired electrical contact with the first electrical connection 22. The desired electrical contact between the latching sleeve 36 and the second electrical connection 23 is made via a second contact section 42 in the guide block 4.

A seal 43 is arranged between the two contact sections 41 and 42, which rests against the second insulation 35 between the end sleeve 30 and the latching sleeve 36. This seal 43 ensures that there is no electrical contact between latching sleeve 36 and end sleeve 30, although moisture can occur in this area. This seal 43 therefore ensures the desired electrical isolation.

In order to be able to remove the electrode 20 from the guide block 4, it is only necessary to press the latching element 40 against the spring force in the direction of the arrow P1 so that the lock is released and the electrode 20 can be removed. For insertion, it is only necessary to insert the electrode 20 into the guide block 4 until the latching element 40 engages.

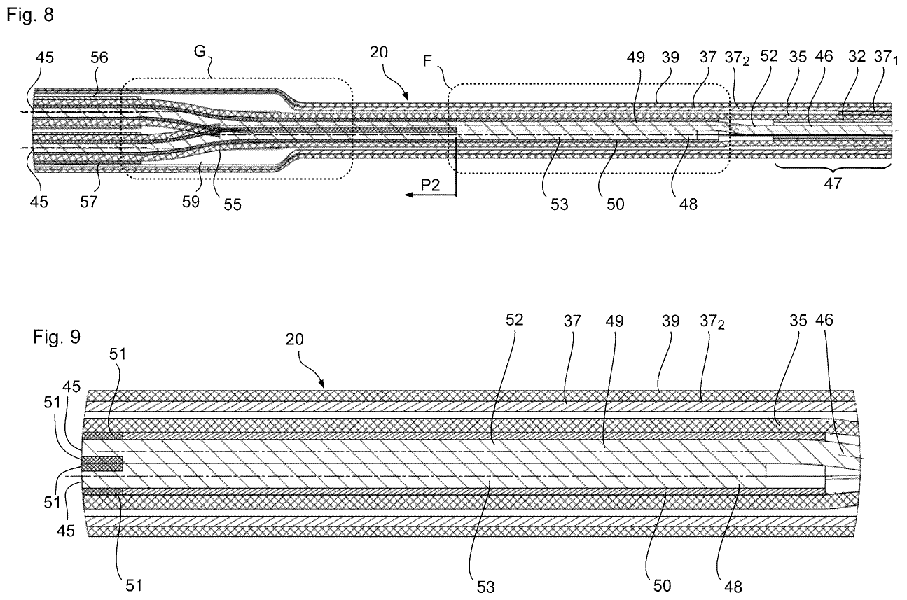

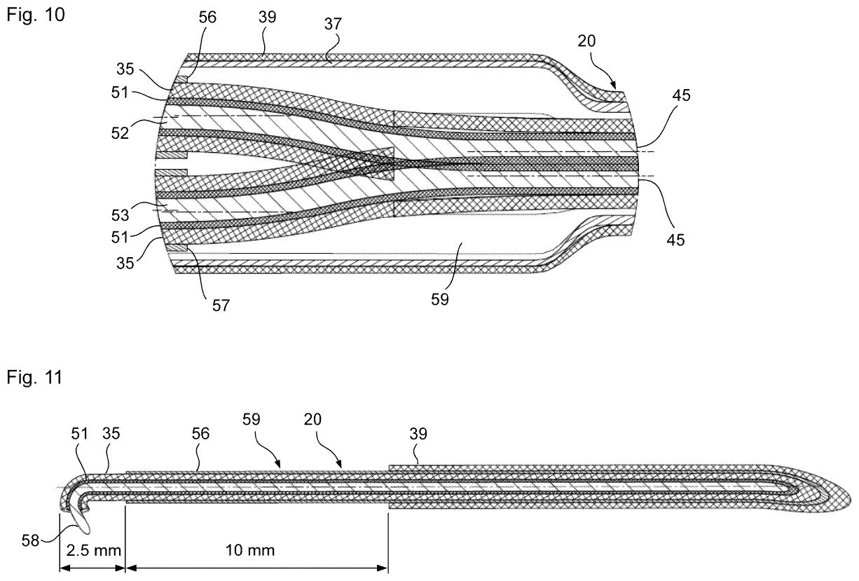

In the top view of the distal end portion 27 shown in FIG. 7, the fork-shaped formation of the distal end portion 27 with two fork arms 59, 60 and a loop 58 of a loop wire 45 is clearly shown. A sectional view along the sectional line C-C (FIG. 3) of the area E (FIG. 7) of the end portion 27 is shown in FIG. 8, wherein the guide 28 is not shown in order to simplify the representation. Detail F of FIG. 8 is shown in FIG. 9 and detail G of FIG. 8 is shown in FIG. 10.

A first proximal end portion 46 of the loop wire 45 is crimped to the inner tube 32 in the region 47. A second proximal end portion 48 ends in front of the region 47 in an axial direction, so that there is an axial distance between the second proximal end portion 48 and the region 47. The second proximal end portion 48 is crimped to a portion 49 of the loop wire 45 coming from the first proximal end portion 46 by means of a connecting sleeve 50 surrounding the second proximal end portion 48 and the portion 49. In the embodiment described here, the connecting sleeve 50 comprises an axial length of 10 mm. The two parts of the loop wire 45, which are crimped by means of the connecting sleeve 50 and run to the distal end 21 and thus to the loop 58, are hereinafter referred to as first and second wire sections 52, 53.

Furthermore, the main tube 37 is formed in two parts, wherein the first part 37₁ of the main tube 37, which ends in the region 47, is pressed together with a second part 37₂ of the main tube 37.

In an axial direction adjacent to the connecting sleeve 50 (indicated by the arrow P2), a third insulation 51 is applied directly to the two wire sections 52, 53 of the loop wire 45, which in turn can be formed as a heat-shrinkable tube (for example PTFE tube or PVDF tube). This third insulation 51 surrounds each of the two loop wire sections 52, 53 from a fork point 55, at which the two wire sections 52, 53 diverge and are thus spaced apart in the radial direction. Thus, the second insulation 35 overlaps the third insulation 51 in the axial direction adjacent to the connecting sleeve 50, whereby possible short circuits between the active electrode 34 and the neutral electrode 38 can be prevented, since liquid can penetrate in the area 59 around the fork point 55 between the main tube 37 and the inner tube 32. After the fork point 55 in an axial direction towards the distal end 21 of the electrode 20, a (first and second) fork tube 56 and 57 is formed around each wire section 52, 53, the main tube 37 being welded to each fork tube 56 and 57 and then terminating slightly to the left of the end of the representation in FIG. 8. However, the first insulation 39 continues and then ends, as can be seen in particular in the sectional view D-D (FIG. 3) of the loop wire section 52 in FIG. 11 and in FIG. 7, approximately 12.5 mm in front of the distal end 21 of the electrode 20. The first and second fork tubes 56, 57 are thus exposed over a length of 10 mm and form the distal end of the neutral electrode 38 or an exposed second end region of the neutral electrode 38. The loop wire 45 is then exposed in the U-shaped region connecting the two fork arms 59, 60 and thus forms the distal end of the active electrode 34 or an exposed first end region of the active electrode 34. The loop wire 45 is preferably formed here from tungsten.

The loop 58 is only one example of a distal working end 21 of the bipolar electrode 20. The distal end 21 may also comprise a cone, a knife, a roller, a roll, etc. Due to the larger radii then present, stainless steel can, but does not have to, be used instead of tungsten, for example. Stainless steel can then also be used for the entire wire 45.

The electrode 20 can thus be referred to as a coaxial bipolar electrode 20, since the neutral electrode 38 runs coaxially to the active electrode 34. Furthermore, both electrodes 34 and 38 are led from the proximal contact portion 25 to the distal end 21 so that, in operation, the current flow takes place from the exposed loop 58 to the exposed end portion of the corresponding fork tube 56 or 57. Thus, in an advantageous manner, no current has to be conducted to or from the patient in this application and the full potential difference is applied directly to the loop 58 and thus to the distal end 21 of the bipolar electrode 20. This ensures, for example, good ignition behavior for the desired plasma in a NaCl solution. Furthermore, the working element 2 itself is advantageously potential-free. The design of the inner tube 32 made of copper is advantageous, as the currents that occur can be conducted well without temperature problems occurring.

What is claimed is:

1. A bipolar electrode for a resectoscope, comprising:
a proximal contact portion;
a middle portion adjoining the proximal contact portion;
a distal end portion adjoining the middle portion;
an active electrode; and
a neutral electrode,
wherein the neutral electrode has a hollow cylindrical shape in the middle portion and coaxially surrounds the active electrode,
wherein the proximal contact portion comprises a first contact of the active electrode and a second contact of the neutral electrode.
wherein the neutral electrode is surrounded by a first insulation from the proximal contact portion to the distal end portion, and
wherein both a first end region of the active electrode and a second end region of the neutral electrode are exposed at the distal end portion such that an electrical voltage applied via the contacts is applied directly between the two end regions.

2. The bipolar electrode according to claim 1, wherein the active electrode comprises a copper conductor in the middle portion.

3. The bipolar electrode according to claim 2, wherein the active electrode comprises a hollow cylindrical conductor in the middle portion.

4. The bipolar electrode according to claim 3, wherein a conductive wire is provided in the hollow cylindrical conductor of the active electrode in the middle portion.

5. The bipolar electrode according to claim 1, wherein the active electrode comprises a hollow cylindrical conductor in the middle portion.

6. The bipolar electrode according to claim 5, wherein a conductive wire is provided in the hollow cylindrical conductor of the active electrode in the middle portion.

7. The bipolar electrode according to claim 1, wherein the active electrode comprises a solid material conductor in the middle portion.

8. The bipolar electrode according claim 1, wherein the active electrode comprises a mechanical reinforcement element in the region of the proximal contact portion.

9. The bipolar electrode according to claim 1, wherein a second insulation is provided between the active electrode and the neutral electrode.

10. The bipolar electrode according to claim 1, wherein the distal end portion is fork-shaped with a first and a second fork arm, and wherein the exposed first end region of the active electrode is located between the first and second fork arms.

11. The bipolar electrode according to claim 1,
wherein a second insulation is disposed between the active electrode and the neutral electrode,
wherein the distal end portion is fork-shaped with a first and a second fork arm,
wherein the exposed first end region of the active electrode is located between the first and second fork arms, and
wherein each of the first and second fork arms comprises a third insulation which is arranged between the active electrode and the neutral electrode and overlaps with the second insulation.

12. The bipolar electrode according to claim 1, wherein the exposed first end region of the active electrode comprises a tungsten wire.

13. The bipolar electrode according to claim 1, wherein the first contact of the active electrode and/or the second contact of the neutral electrode comprises a metallic sleeve.

14. The bipolar electrode according to claim 1, wherein the first contact of the active electrode and/or the second contact of the neutral electrode comprises a metallic coating.

15. The bipolar electrode according to claim 1, wherein the second contact of the neutral electrode is configured such that the second contact can be brought into engagement with a latching connection.

16. A resectoscope, comprising the bipolar electrode according to claim 1.

17. The resectoscope according to claim 16, further comprising a guide block with a latching element which, in the inserted state of the bipolar electrode, effects a latching connection with the first contact of the active electrode or the second contact of the neutral electrode.

18. The resectoscope according to claim 17, further comprising:
a guide block with a first contact portion for the first contact of the active electrode; and
a second contact portion for the second contact of the neutral electrode,
wherein a seal is arranged between the two contact portions, and
wherein the seal, in the inserted state of the bipolar electrode, prevents a liquid from being able to pass along the proximal contact portion from the second contact to the first contact.

19. The resectoscope according to claim 16, further comprising:
a guide block with a first contact portion for the first contact of the active electrode; and
a second contact portion for the second contact of the neutral electrode,
wherein a seal is arranged between the two contact portions, and
wherein the seal, in the inserted state of the bipolar electrode, prevents a liquid from being able to pass along the proximal contact portion from the second contact to the first contact.
